(19) United States
(12) Patent Application Publication
(10) Pub. No.: US 2023/0315099 A1
(43) Pub. Date: Oct. 5, 2023

(54) PROGRAM, CLEANING DEVICE, AND INFORMATION PROCESSING METHOD

(71) Applicant: H.U. GROUP RESEARCH INSTITUTE G.K., Tokyo (JP)

(72) Inventors: Takeyuki KOTAKA, Tokyo (JP); Tatsuki MATSUNO, Tokyo (JP); Kazuya OMI, Tokyo (JP)

(73) Assignee: H.U. GROUP RESEARCH INSTITUTE G.K., Tokyo (JP)

(21) Appl. No.: 18/018,420

(22) PCT Filed: Jun. 28, 2021
(86) PCT No.: PCT/JP2021/024266

§ 371 Date: Jan. 27, 2023

(30) Foreign Application Priority Data
Jul. 29, 2020 (JP) 2020-128466

(51) Int. Cl. G05D 1/02; G06V 40/20; G06V 10/58; G06V 20/52; G16H 40/20; A47L 11/40; A47L 9/28; A61L 2/24; A61L 2/10

(52) U.S. Cl. CPC G05D 1/0212; G06V 40/20; G06V 10/58; G06V 20/52; G16H 40/20; A47L 11/4011; A47L 9/2852; A47L 9/2805; A61L 2/24; A61L 2/10; G05D 1/0246; G06T 2207/10036; A47L 2201/04; A47L 2201/06; A61L 2202/11; A61L 2202/17; A61L 2202/14; A61L 2202/25; A61L 2202/16; G05D 2201/0203

(57) ABSTRACT

Provided are a program and the like in which a cleaning efficiency can be improved.

A program allows a computer to execute processing of: acquiring an image including a still image or a moving image that is obtained by imaging an inside of a medical facility with an imaging device provided in the medical facility, or operation content information relevant to operation content in the medical facility; specifying a part to be cleaned in the medical facility and a cleaning method, on the basis of the acquired image or the acquired operation content information; and outputting a cleaning command according to the specified part to be cleaned in the medical facility and the specified cleaning method.

### REFERENCE TO RELATED APPLICATIONS

This nonprovisional application is a continuation of International Application No. PCT/JP2021/024266, which was filed on Jun. 28, 2021, and which claims priority to Japanese Patent Application No. 2020-128466, which was filed in Japan on Jul. 29, 2020, and which are both herein incorporated by reference.

### BACKGROUND OF THE INVENTION

### Field of the Invention

In the related art, a so-called autonomous cleaning device that cleans the floor while autonomously traveling on the floor as a surface to be cleaned is known (for example, Japanese Patent Laid-Open Publication No. 2002-360480). In addition, recently, an autonomous traveling body having a function of creating a map according to the surrounding environment, which is an area where the autonomous traveling body moves, has been developed.

### Description of the Background Art

In Japanese Patent Laid-Open Publication No. 2014-219723, an autonomously movable cleaning robot having a teaching traveling mode of generating a map representing an area as a function, in which the cleaning robot autonomously travels by a manipulator manually manipulating the cleaning robot, is disclosed. According to Japanese Patent Laid-Open Publication No. 2014-219723, the cleaning robot sets a suitable traveling schedule in the teaching traveling mode, and thus, is capable of completing autonomous travel according to the traveling schedule, in a reproductive traveling mode.

### SUMMARY OF THE INVENTION

However, in the cleaning robot disclosed in Japanese Patent Laid-Open Publication No. 2014-219723, the entire cleaning target area is cleaned on the basis of map information, and thus, a clean part is also cleaned, which is not efficient.

An object of the present disclosure is to provide a program and the like in which a cleaning efficiency can be improved.

A program according to one aspect of the present disclosure allows a computer to execute processing of: acquiring an image including a still image or a moving image that is obtained by imaging an inside of a medical facility with an imaging device provided in the medical facility, or operation content information relevant to operation content in the medical facility; specifying a part to be cleaned in the medical facility and a cleaning method, on the basis of the acquired image or the acquired operation content information; and outputting a cleaning command according to the specified part to be cleaned in the medical facility and the specified cleaning method.

According to the present disclosure, a cleaning efficiency can be improved.

### BRIEF DESCRIPTION OF THE DRAWINGS

The above and further objects and features will more fully be apparent from the following detailed description with accompanying drawings.

FIG. 1 is an explanatory diagram illustrating an outline of a cleaning system in a first embodiment.

FIG. 2 is a block diagram illustrating a configuration of a cleaning device.

FIG. 3 is an explanatory diagram illustrating a method of specifying a part to be cleaned and a cleaning method.

FIG. 4 is a flowchart illustrating an example of a processing procedure that is executed by the cleaning device.

FIG. 5 is a block diagram illustrating a configuration of a cleaning device in a second embodiment.

FIG. 6 is a schematic view of a learning model.

FIG. 7 is a flowchart illustrating an example of a processing procedure that is executed by the cleaning device in the second embodiment.

FIG. 8 is a block diagram illustrating a configuration of a cleaning device in a third embodiment.

FIG. 9 is a flowchart illustrating an example of a processing procedure that is executed by the cleaning device in the third embodiment.

The present disclosure will be described in detail with reference to the drawings illustrating embodiments thereof.

### DETAILED DESCRIPTION

### First Embodiment

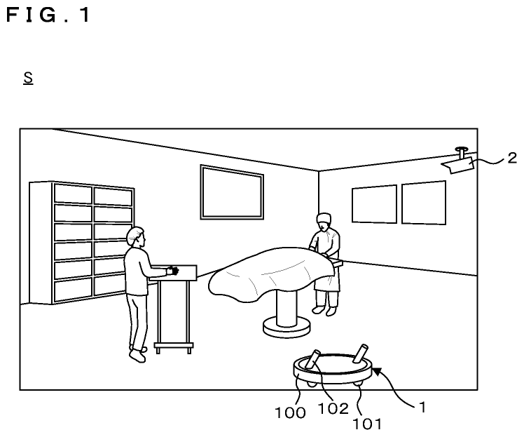

FIG. 1 is an explanatory diagram illustrating the outline of a cleaning system S in a first embodiment. The cleaning system S includes a cleaning device 1 cleaning an inside of a medical facility, and an imaging device 2 imaging the inside of the medical facility.

The cleaning device 1 includes a main body 100 configuring an outer shell, a movement mechanism 101 for allowing the main body to move, and a cleaning mechanism 102 for cleaning a surface to be cleaned, and cleans the inside of the medical facility while automatically moving in the medical facility. The main body 100, for example, is a columnar housing.

The imaging device 2, for example, is a camera. The imaging device 2 is provided in the medical facility, and images a moving image or a still image of an imaging target including goods or a medical service worker in the medical facility. The imaging device 2 includes an imaging element such as a charge coupled device (CCD) image sensor, a lens, and the like, and performs photoelectric conversion to light incident through the lens with the imaging element to be output as image data. The imaging device 2 may be a stereo camera including a plurality of imaging elements. A plurality of imaging devices 2 may be provided, and the entire inside of the medical facility may be imaged from various angles. The imaging device 2 has a communication function, and transmits image data that is a moving image or a periodically imaged still image to the cleaning device 1. The imaging device 2 is not limited to being provided in the medical facility, and may be provided in the cleaning device 1.

In the medical facility, a temperature sensor detecting temperature information, a sound collecting microphone detecting a sound, or the like may further be provided to detect various information pieces in the medical facility may be detected.

In the cleaning system S, a contamination state with respect to an operation that is actually performed in the medical facility is derived on the basis of an image that is obtained by imaging the operation in the medical facility, or operation content information relevant to operation content, and the cleaning device 1 automatically cleans the inside of the medical facility, on the basis of a part to be cleaned and a cleaning method according to the contamination state. The medical facility to which the cleaning system S is applied, for example, is a hospital. In this embodiment, the medical facility, for example, may be each room such as an operating room, an examination room, and a waiting room in the hospital, or may be the entire facility including a plurality of rooms. Note that, this cleaning system S is not limited to the medical facility, and for example, can be applied to a facility such as a research station and a laboratory.

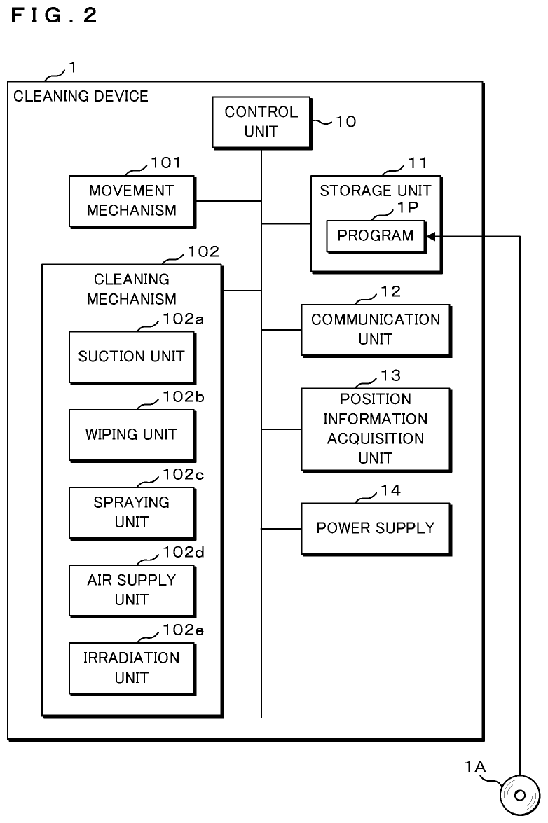

FIG. 2 is a block diagram illustrating the configuration of the cleaning device 1. The cleaning device 1 includes a control unit 10, a storage unit 11, a communication unit 12, a position information acquisition unit 13, a power supply 14, and the like, in addition to the movement mechanism 101 and the cleaning mechanism 102 described above.

The control unit 10 is a processor using one or a plurality of central processing units (CPU) or graphics processing units (GPU), and executes processing by controlling each constituent using a built-in memory such as a read only memory (ROM) and a random access memory (RAM).

The storage unit 11 includes a storage device such as a flash memory, a hard disk, and a solid state drive (SSD). The storage unit 11 stores a program and data including a program 1P that are referred to by the control unit 10. The control unit 10 executes processing relevant to automatic cleaning described below, on the basis of the program 1P.

The program 1P stored in the storage unit 11 may be provided by a non-transitory recording medium 1A in which the program 1P is recorded to be readable. The recording medium 1A, for example, is a portable memory such as a CD-ROM, a universal serial bus (USB) memory, a secure digital (SD) card, a micro-SD card, and compact flash (Registered Trademark). In this case, the control unit 10 reads out the program 1P from the recording medium 1A by using a reader that is not illustrated, and stores the read program 1P in the storage unit 11. In addition, the program 1P stored in the storage unit 11 may be provided by communication through the communication unit 12. In this case, the control unit 10 acquires the program 1P through the communication unit 12, and stores the acquired program 1P in the storage unit 11. Note that, the storage unit 11 may include a plurality of storage devices, or may be an external storage device connected to the cleaning device 1.

The communication unit 12, for example, is Bluetooth (Registered Trademark), Wi-Fi (Registered Trademark), and other wireless local area networks (LAN), and a wireless communication device based on a communication standard, such as 4G, 5G, and long term evolution (LTE). The control unit 10 communicates with the imaging device 2 through the communication unit 12, and acquires the image.

The position information acquisition unit 13 acquires position information of the cleaning device 1 in the medical facility. The position information acquisition unit 13, for example, includes a global positioning system (GPS) receiver, a receiver of a beacon signal, an ultrasonic sensor, a radar sensor, an image sensor, laser imaging detection and ranging (LIDAR), a gyroscope sensor, an acceleration sensor, or the like. The position information acquisition unit 13 may include a rotary encoder or the like that detects a traveling speed or a traveling distance of the cleaning device 1 by detecting the number of rotations of a motor of the movement mechanism detection.

The power supply 14, for example, includes a secondary battery such as a lithium-ion battery, and supplies power to each functional element of the cleaning device 1. The power supply 14 includes a connection terminal, and the power is supplied to the secondary battery by the connection terminal being connected to a connection terminal provided in an external power feeder (not illustrated).

The movement mechanism 101, for example, includes a wheel, a motor, a gear, and the like. Power that is supplied by the motor is transferred to the wheel by the gear, and the wheel is in contact with the surface to be cleaned to generate a driving force, and moves on the surface to be cleaned.

The cleaning mechanism 102, for example, includes a suction unit 102a, a wiping unit 102b, a spraying unit 102c, an air supply unit 102d, an irradiation unit 102e, and the like. Each constituent, for example, is provided on the bottom surface or the lateral surface of the main body, and cleans the surface to be cleaned that is the floor in the medical facility.

The suction unit 102a, for example, includes a suction motor and a suction port. The suction unit 102a sucks and collects dust such as collected dust together with the air from the suction port by the driving of the suction motor. The wiping unit 102b, for example, wipes the surface to be cleaned by using a wiping cloth or the like. The spraying unit 102c includes a chemical tank and a spraying port, and sprays chemicals that are supplied from the chemical tank toward the surface to be cleaned from the spraying port. Examples of the chemicals dispersed by the spraying unit 102c include a hypochlorous acid, ethanol, or the like. The spraying unit may include a plurality of chemical tanks, and may spray chemicals according to contamination content of the surface to be cleaned. The air supply unit 102d sends the air to the surface to be cleaned. The air supply unit 102d may include a heater and the like, and may send the air that is heated by the heater to dry the surface to be cleaned. The irradiation unit 102e includes a sterilization lamp that emits an ultraviolet ray to the surface to be cleaned, and sterilizes various bacteria of the surface to be cleaned. The cleaning device 1 cleans and sterilizes the surface to be cleaned by using each constituent of the cleaning mechanism 102 described above.

Each constituent of the cleaning mechanism 102 described above is not limited to being provided on the bottom surface or the lateral surface of the main body. The cleaning device 1, for example, may include an arm, and may clean the wall surface or the ceiling, or arranged objects such as a bed or a desk in the medical facility by the constituent of the cleaning mechanism 102 that is provided on the tip of the arm. That is, the surface to be cleaned may include not only a two-dimensional plane but also a three-dimensional space. In this case, it is preferable that the position information acquisition unit 13 acquires position information of each constituent of the cleaning mechanism 102, in addition to the position information of the cleaning device 1.

In the cleaning system S configured as described above, the part to be cleaned and the cleaning method according to the state in the medical facility are specified, and the part to be cleaned is cleaned by the specified cleaning method, in the cleaning in the medical facility. The cleaning device 1 specifies a suitable part to be cleaned and a suitable cleaning method, in accordance with the state in the medical facility, and thus, efficiently cleans the inside of the medical facility.

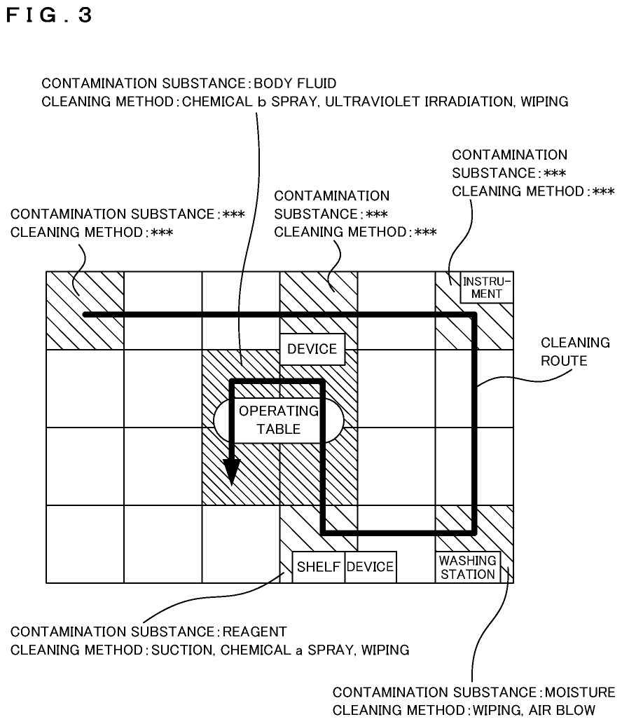

The part to be cleaned and the cleaning method, for example, is specified on the basis of the image that is obtained by imaging the operation in the medical facility, the operation content information relevant to the operation content in the medical facility, and the like. More specifically, the cleaning device 1 specifies the contamination state in the medical facility, on the basis of the image, the operation content information, or the like, and determines the part to be cleaned and the cleaning method in accordance with the specified contamination state. FIG. 3 is an explanatory diagram illustrating a method of specifying the part to be cleaned and the cleaning method. Specifying the part to be cleaned and the cleaning method will be described in detail by using FIG. 3.

In the image to be a specifying element of the part to be cleaned and the cleaning method, the inside of the medical facility is set to an imaging area, and the goods or the medical service worker in the medical facility are included in the image that is imaged. The control unit 10 of the cleaning device 1, for example, extracts the goods or the medical service worker included in the image by using a method such as pattern matching, on the basis of a feature amount of the image and a feature amount of the goods or the medical service worker that is stored in advance. The control unit 10 specifies the position of the goods in the medical facility, the type of goods, the position of the medical service worker, the type of medical service worker, and the like, on the basis of an extraction result. The control unit 10 may determine the goods or the medical service worker that are included in the image, by using other mechanical learning method. The control unit 10 executes the processing described above with respect to each frame of the acquired moving image or each of a plurality of still images.

The control unit 10 derives the contamination state including a contamination range in the medical facility and the type of contamination substance, on the basis of the acquired data. The control unit 10 stores in advance MAP information of the medical facility. As illustrated in FIG. 3, the inside of the medical facility is divided into a plurality of areas, on the basis of positional coordinates, and the control unit 10 generates contamination state MAP associated with the contamination state in each of the areas.

The control unit 10 calculates each contamination point in an area including the corresponding position on MAP, on the basis of information of the position of the goods or the medical service worker that are extracted from the image. Since the contamination substance is likely to be attached to the area including the position in which the medical service worker is extracted, the contamination point is applied to the area. In this case, for example, weighting according to the type of medical service worker may be performed. For example, in a case where a doctor is extracted, a higher contamination point is applied compared to a case where a clinical nurse is extracted. The control unit 10 calculates the total value of the contamination points with respect to each of the acquired images. As the length of a staying time of the medical service worker with respect to the same part increases, the total value of the contamination points increases. Similarly, since the contamination substance is likely to be attached to the area including the position in which the goods used in the operation, such as an operating table, a washing station, and a device, are extracted, the contamination point is applied to the area. The control unit 10 derives the area to which the contamination point is applied or an area in which the contamination point is a threshold value or more as the contamination range. The control unit 10 may derive a contamination degree indicating the degree of contamination of the contamination range, on the basis of the contamination point. In FIG. 3, the area that is specified as the contamination range is represented by applying different hatching in accordance with the contamination degree. Note that, in general, position information of goods arranged in a fixed position may also be acquired on the basis of the MAP information.

In addition, the control unit 10 derives the type of contamination substance that is attached to the contamination range, on the basis of the type of goods corresponding to the contamination range. For example, in a case where the goods are an operating table, it is determined that the contamination substance includes the body fluid (blood). In a case where the goods are a washing station, it is determined that the contamination substance includes the moisture. The control unit 10 may specify the type of goods, on the basis of the label of medicinal products in the image, and may determine the type of contamination substance. According to the processing described above, the control unit 10 specifies the contamination state including the contamination range and the type of contamination substance, on the basis of the image. For example, in an example illustrated in FIG. 3, it is specified that a contamination state of four areas including an operating table illustrated in the vicinity of the center of the medical facility is the contamination range to which a high contamination point is applied, and the type of contamination substance includes the body fluid.

The control unit 10 determines the part to be cleaned and the cleaning method, on the basis of the specified contamination state. The control unit 10 specifies a part to be intensively cleaned in the medical facility, a cleaning means for the part to be intensively cleaned, the type of chemicals to be used, and the like, on the basis of the contamination range in the medical facility and the type of contamination substance in the contamination range. The part to be intensively cleaned is determined on the basis of the contamination range. An area corresponding to the contamination range illustrated by hatching in FIG. 3 is determined as the part to be intensively cleaned. The cleaning means is determined on the basis of the type of contamination substance, the shape and the material of the part to be intensively cleaned, and the like, and for example, one or a plurality of means are selected from suction, wiping, chemical spray, air blow (air supply), ultraviolet irradiation, or the like. The chemicals to be used are determined on the basis of the type of contamination substance, the material of the part to be intensively cleaned, and the like. The control unit 10 specifies the cleaning method with reference to a table in which the contamination state and the cleaning method stored in advance are associated with each other. For example, in the example illustrated in FIG. 3, four areas including the operating table illustrated in the vicinity of the center of the medical facility are the part to be intensively cleaned, and chemical b spray, ultraviolet irradiation, and wiping are selected as the cleaning method.

In addition, the control unit 10 specifies a cleaning route in the medical facility, on the basis of the specified part to be intensively cleaned, and the MAP information in the medical facility that is stored in advance. The cleaning device 1 may clean the entire inside of the medical facility, or may clean only the part to be intensively cleaned. In a case of cleaning the entire inside of the medical facility, basic cleaning such as suction may be performed in parts other than the specified part to be intensively cleaned, and intensive cleaning according to the specified cleaning method may be performed in the part to be intensively cleaned. The control unit 10, for example, derives a route connecting a plurality of parts to be cleaned that are a cleaning target in the shortest distance, as the cleaning route. In the example of FIG. 3, a route connecting each of the parts to be intensively cleaned in order illustrated by an arrow is derived as the cleaning route. Note that, the control unit 10 is not limited to using a rule-based method, and the part to be cleaned and the cleaning method may be determined by using other mechanical learning method.

The control unit 10 may use operation content information relevant to the operation content in the medical facility, as the specifying element of the part to be cleaned and the cleaning method. The operation content information, for example, is medical chart data in which the operation in the medical facility is recorded. In the medical chart data, information relevant to the operation in the medical facility is recorded, and for example, operating content, an operating time, the amount of bleeding of a patient, and the like are included. Operation content relevant to measurement, or the goods used, such as a thermometer or a blood-pressure gauge, may be specified from a measurement time of a body temperature, a blood pressure, or the like that is included in the medical chart data. The control unit 10 derives the contamination state, on the basis of the acquired medical chart data. For example, the control unit 10 specifies an operation range of the medical service worker, the operation content, the type of medical service worker performing the operation, and the like, on the basis of the operating content, and derives the contamination range in the medical facility or the type of contamination substance, on the basis of the specified data. The control unit 10 derives the contamination range or the type of contamination substance, on the basis of the position or the type of goods such as devices to be used and chemicals to be used, which is specified on the basis of the operating content. The type of contamination substance is derived on the basis of the presence or absence of the bleeding of the patient (the amount of bleeding).

For example, in a case where the operation content is specified as an operative treatment along with bleeding from the medical chart data, it is specified that the operating table is included in the operation range, and a primary doctor of the operative treatment and an assistant are included in the type of medical service worker. In this case, it is determined that a predetermined contamination point is applied to an area including the operating table, and the contamination substance with respect to the area includes the body fluid. In a case where it is specified that a specific device is used from the operating content, the contamination point is applied to an area including the device. The control unit 10 may add the contamination point, on the basis of the operating time and the number of medical service workers involved in the operation. For example, it is determined that the degree of contamination increases as the operating time increases and the number of people performing the operation increases, and more contamination points are applied.

The control unit 10 may further use environment information such as the temperature, the humidity, and the hours of daylight in the medical facility, as the specifying element of the part to be cleaned and the cleaning method. The control unit 10 may further use device information such as the current location of the cleaning device 1, the type and the remaining amount of loaded chemicals, a remaining battery level, and the type of ultraviolet lamp. The control unit 10 may also use a detected value of a detection device such as a thermosensor and a sound collecting microphone provided in the medical facility. As described above, in the cleaning system S specifies the part to be cleaned and the cleaning method, in accordance with the usage of the medical facility, on the basis of the operation content of the medical service worker in the medical facility, the type of used goods, or the like. By cleaning a part required to be cleaned with an effective means, it is possible to improve a cleaning efficiency.

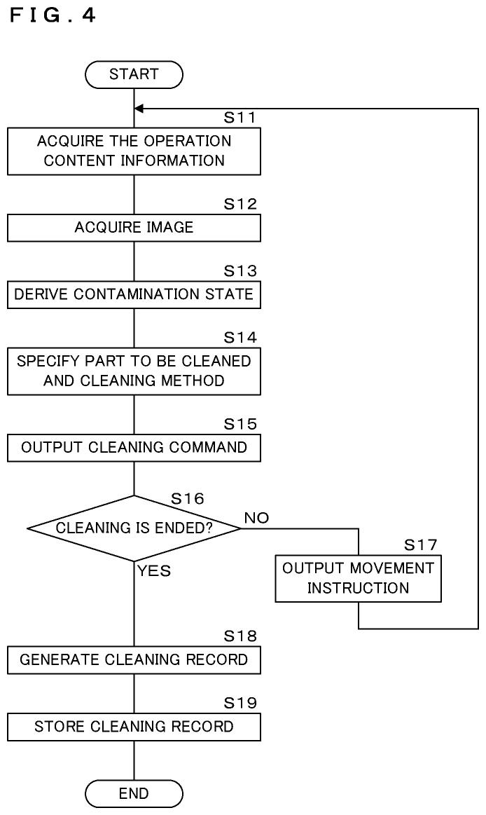

FIG. 4 is a flowchart illustrating an example of a processing procedure that is executed by the cleaning device 1. The control unit 10 of the cleaning device 1 executes the following processing, in accordance with the program 1P. The control unit 10, for example, acquires a cleaning start instruction or the like from the outside and starts the following processing. The control unit 10 may execute the following processing at a predetermined timing, on the basis of a cleaning schedule stored in advance.

The control unit 10 of the cleaning device 1 acquires the operation content information associated with identification information of the medical facility that is an operation target (step S11). The control unit 10, for example, communicates with a server of the medical facility, thereby acquiring the medical chart data as the operation content information. In this case, the control unit 10 specifies the medical facility that is the cleaning target, on the basis of the identification information of the medical facility according to the cleaning start instruct, and acquires a plurality of medical chart data pieces relevant to each operation performed until the current cleaning is started after the previous cleaning is ended, in the specified medical facility. The control unit 10 may further acquire environment information, the device information, and the detected value.

The control unit 10 acquires the image that is obtained by imaging the inside of the medical facility from the imaging device 2 (step S12). In this case, the control unit 10 acquires the moving image or the plurality of still images that are obtained by imaging each of the operations performed until the current cleaning is started after the previous cleaning is ended, in the medical facility that is the cleaning target. Note that, every time when the imaging device 2 images a new image, image information may be transmitted from the imaging device 2, and may be acquired by the cleaning device 1. The control unit 10 transitorily stores the acquired operation content information and the acquired image data in association with each other.

The control unit 10 derives the contamination state including the contamination range in the medical facility and the type of contamination substance, on the basis of the acquired operation content information and the acquired image (step S13). Specifically, the control unit 10 calculates the contamination points in each of the areas in the medical facility, on the basis of the operation range, the operation content, the goods used, and the like to be specified by each of the medical chart data pieces, and derives the contamination range, on the basis of the total point of the contamination points. Further, the control unit 10 derives the type of contamination substance with respect to each of the areas. In addition, the control unit 10 extracts the goods or the medical service worker included in the image, and specifies the position or the type of goods, the position or the type of medical service worker, and the like. The control unit 10 calculates the contamination point in each of the areas in the medical facility, on the basis of the specified position or the specified type of goods, the specified position or the specified type of medical service worker, and the like, and derives the contamination range, on the basis of the total point of the contamination points. Further, the control unit 10 derives the type of contamination substance with respect to each of the areas. The control unit 10 may acquire the degree of contamination. The control unit 10 may derive the contamination state, on the basis of any one of the operation content information and the image.

The control unit 10 specifies the part to be cleaned and the cleaning method in the medical facility, on the basis of the derived contamination state (step S14). Specifically, the control unit 10 specifies the part to be intensively cleaned or the part to be cleaned including the cleaning route, on the basis of the contamination range. In addition, the control unit 10 specifies cleaning methods for parts to be intensively cleaned, respectively, with reference to a table in which the type of contamination substance and the shape and the material of the part to be intensively cleaned are associated with the cleaning means or the type of chemicals to be used.

The control unit 10 outputs a cleaning command for cleaning the part to be cleaned by the specified cleaning method to each constituent (step S15). The movement mechanism 101 travels the travels the cleaning device 1 along the cleaning route. The cleaning mechanism 102 cleans each of the parts to be cleaned that are a target, in accordance with the cleaning method. In this case, the control unit 10 may create map data while assuming a self-position by a technology such as simultaneous localization and mapping (SLAM), and may autonomously move.

The control unit 10 determines whether or not the cleaning is ended (step S16). In a case where it is determined that the cleaning is not ended by acquiring a cleaning command according to a second medical facility (step S16: NO), the control unit 10 outputs a movement instruction for the second medical facility to the movement mechanism 101 (step S17). After moving to the second medical facility, the control unit 10 returns the processing to step S11, and executes loop processing. The control unit 10 acquires an image of the imaging device 2 provided in the second medical facility, and operation content information in the second medical facility, outputs the cleaning command, in accordance with the specified part to be cleaned and the specified cleaning method, on the basis of the acquired information, and executes cleaning in the second medical facility.

On the other hand, in a case where it is determined that the cleaning is ended by not acquiring the cleaning command according to the second medical facility (step S16: YES), the control unit 10 generates a cleaning record (step S18). In the cleaning record, information relevant to the cleaning performed by the cleaning device 1 is recorded, and for example, a cleaning time, the part to be cleaned, the cleaning method, and the like in the medical facility are included. The control unit 10 stores the generated cleaning record in the storage unit 11 (step S19), and ends a set of processing. The cleaning device 1 may return to a power feeding station including a power feeder or the like after the cleaning, power feeding, and may replenish the chemicals, or the like.

In the processing described above, the control unit 10 may transmit the cleaning record to the server of the medical facility, or the like. A manager of the medical facility is capable of reliably recognizing a cleaning result, on the basis of the received cleaning record.

In the processing described above, the control unit 10 may acquire the image imaged by the imaging device 2 or the operation content information in real time during the operation, and may frequently output the cleaning command, in accordance with the contamination state that is derived from the acquired image or the acquired operation content information. For example, in a case where the cleaning target is a waiting room, a nurse station, or the like, it is preferable that cleaning such as the sterilization of the device is executed at a suitable timing, in accordance with the contamination state that is derived from the usage of the device, or the like when the operation is consecutively executed. In a tablet device, a blood-pressure gauge, or the like, a threshold value of the contamination degree is set, and in a case where the contamination degree is the predetermined value or more, the cleaning command is output, and thus, the monitoring of the contamination state and the cleaning can be executed in parallel.

According to the processing described above, the cleaning device 1 derives the contamination state for each operation in the medical facility that is the cleaning target, on the basis of an image that is obtained by imaging an operation state in the medical facility, and the operation content. The cleaning device 1 specifies the part to be cleaned and the cleaning method according to the contamination state, and performs automatic cleaning while autonomously moving. The cleaning device 1 specifies the part to be cleaned and the cleaning method, on the basis of the actual contamination state, and thus, is capable of efficiently and effectively performing the cleaning. In addition, the cleaning device 1 avoids excessive cleaning with respect to the surface to be cleaned, and thus, is capable of preventing the surface to be cleaned from being degraded and of efficiently using power, chemicals, or the like.

### Second Embodiment

In a second embodiment, a configuration will be described in which the operation state of the medical service worker is assumed by using a learning model. Hereinafter, differences between the second embodiment and the first embodiment will be described. Since configurations except for the following configurations are the same as those of the cleaning system S of the first embodiment, the same reference numerals will be applied to the common configurations, and the detailed description thereof will be omitted.

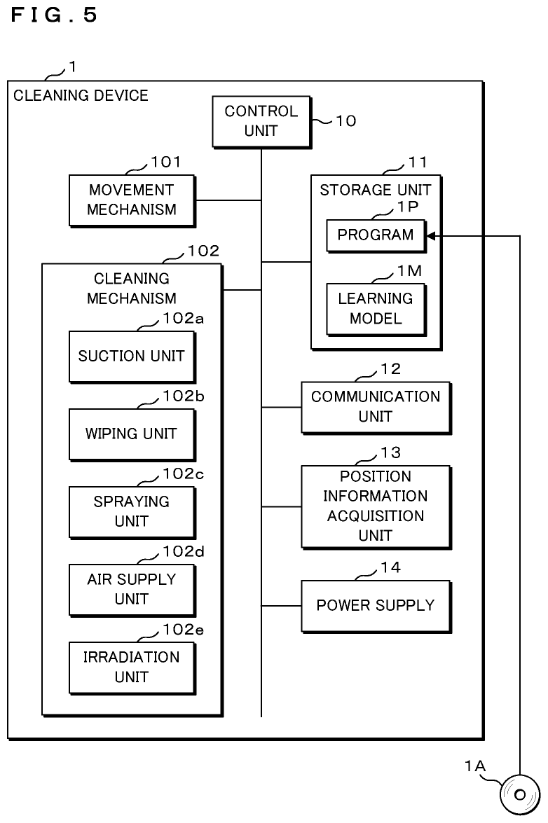

FIG. 5 is a block diagram illustrating the configuration of the cleaning device 1 in the second embodiment. In the storage unit 11 of the cleaning device 1, a learning model 1M is stored. The learning model 1M is a learning model that is used in the processing of assuming the operation state of the medical service worker in the medical facility, on the basis of the image from the imaging device 2. The learning model 1M is described by definition information thereof. The definition information of the learning model 1M includes structure information of the learning model 1M, various parameters such as the weight and the bias between nodes to be used in the learning model 1M, and the like. In this embodiment, the learning model 1M that is learned in advance by a predetermined learning algorithm is stored in the storage unit 11, using the image that is imaged by the imaging device 2, and label data indicating the operation state of the medical service worker, as training data.

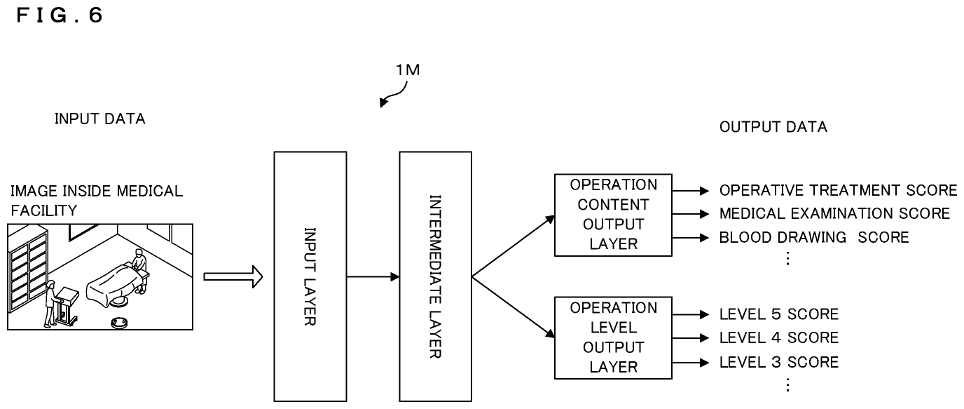

FIG. 6 is a schematic view of the learning model 1M. The learning model 1M is generated and learned in advance in the cleaning device 1 or an external device, by deep learning using a neural network. The learning algorithm, for example, is convolution neural network (CNN).

The learning model 1M includes an input layer for inputting the image that is imaged by the imaging device 2 and includes the medical service worker performing the operation in the medical facility, an output layer for outputting the information indicating the operation state of the medical service worker, and an intermediate layer (hidden layer) for extracting the feature amount. The intermediate layer includes a plurality of nodes for extracting a feature amount of input data, and delivers the extracted feature amount to the output layer by using various parameters. In a case where the image data is input to the input layer, an arithmetic operation is performed in the intermediate layer by learned parameters, and output information relevant to the operation state is output from the output layer.

The output data that is output from the output layer of the learning model 1M is the operation state of the medical service worker. The operation state is information relevant to the operation state of the medical service worker in the image, and for example, includes the operation content, an operation level, and the like of the medical service worker. In the operation content, for example, an operative treatment, medical examination, blood drawing, and the like are included. The operation level indicates the intensity of the operation, and for example, is sorted into five stages of 1 to 5, in accordance with the degree of performance of the medical service worker. The operation state is intense as the numerical value of the operation level increases.

The learning model 1M includes a plurality of output layers for respectively outputting the operation content and the operation level as the operation state. The output layer for outputting the operation content includes nodes respectively corresponding to operation content pieces that are set, and outputs an accuracy with respect to each of the operation content pieces as a score. The cleaning device 1 is capable of setting the operation content with the highest score or the operation content of which the score is a threshold value or more, as an output value of the output layer for outputting the operation content. Note that, the output layer may include one output node for outputting the operation content with the highest accuracy, instead of including a plurality of output nodes for outputting the accuracy of each of the operation content pieces. The output value indicating the numerical value of the operation level is output from the output layer of the operation level. As described above, the image including the medical service worker performing the operation in the medical facility is input, the learning model 1M outputs the information indicating the operation state.

The control unit 10 collects in advance an information group in which the known operation state is applied to a lot of images collected in the past, as the training data, and learns the learning model 1M. In the operation state, for example, data obtained from the medical chart data corresponding to the operation, determination performed by the person performing the operation, or the like may be set as a ground truth label. The control unit 10 learns various parameters, the weight, and the like that configure the learning model 1M to output the operation state according to the image output, for example, by using a backpropagation method.

The learning model 1M is not limited to CNN. The learning model 1M may be a recurrent neural network (RNN) in a case of acquiring time-series data. The learning model 1M may be a learning model constructed by other learning algorithms such as a support vector machine and a regression tree, not using a neural network.

The control unit 10 specifies the contamination state in the medical facility, on the basis of an assumption result of the learning model 1M described above. For example, the operation range of the medical service worker, a medical tool and a medicinal product to be used, the presence or absence of bleeding, and the like are different in accordance with the operation content. In the medical facility where the operative treatment along with bleeding is performed, the blood is likely to be attached in the vicinity of the operating table. In addition, since the medical service worker intensively moves the body as the operation level increases, a large amount of body fluid or medicinal product is attached, and an attached range is likely to broaden. From such a viewpoint, the control unit 10 specifies the contamination state in the medical facility, on the basis of the assumption result of the operation state by the learning model 1M, and acquires the part to be cleaned in the medical facility and the cleaning method after the operation is ended.

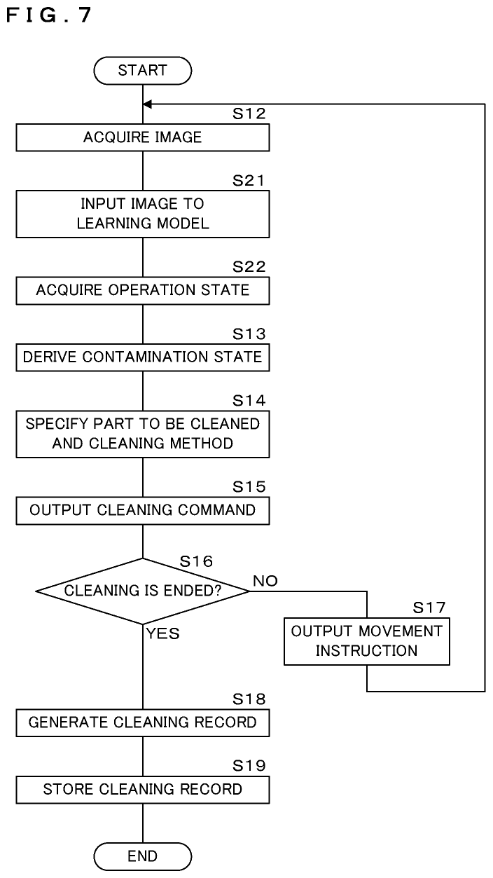

FIG. 7 is a flowchart illustrating an example of a processing procedure that is executed by the cleaning device 1 in the second embodiment. The same step numbers will be applied to the processing common to FIG. 4 of the first embodiment, and the detailed description thereof will be omitted.

The control unit 10 of the cleaning device 1 acquires the image that is obtained by imaging the inside of the medical facility from the imaging device 2 (step S12), and transitorily stores the image.

The control unit 10 inputs the acquired image to the learning model 1M (step S21), and acquires the operation state that is output from the learning model 1M (step S22). The control unit 10 derives the contamination state including the contamination range in the medical facility, the degree of contamination, the type of contamination substance, and the like, on the basis of the operation state including the operation content and the operation level (step S13).

The control unit 10 specifies the part to be cleaned in the medical facility and the cleaning method, on the basis of the derived contamination state (step S14). Hereinafter, as with the first embodiment, the control unit 10 executes the processing of step S15 to step S19, and ends a set of processing.

According to this embodiment, the cleaning device 1 is capable of accurately assuming the operation state from the image data by using the learning model 1M. Since the cleaning is performed on the basis of the part to be cleaned and the cleaning method that are suitably determined in accordance with the operation state, a cleaning efficiency can be further improved.

### Third Embodiment

In a third embodiment, a configuration will be described in which the contamination state in the medical facility is specified by using a hyperspectral camera. Hereinafter, differences between the third embodiment and the first embodiment will be described. Since configurations except for the following configurations are the same as those of the cleaning system S of the first embodiment, the same reference numerals will be applied to the common configurations, and the detailed description thereof will be omitted.

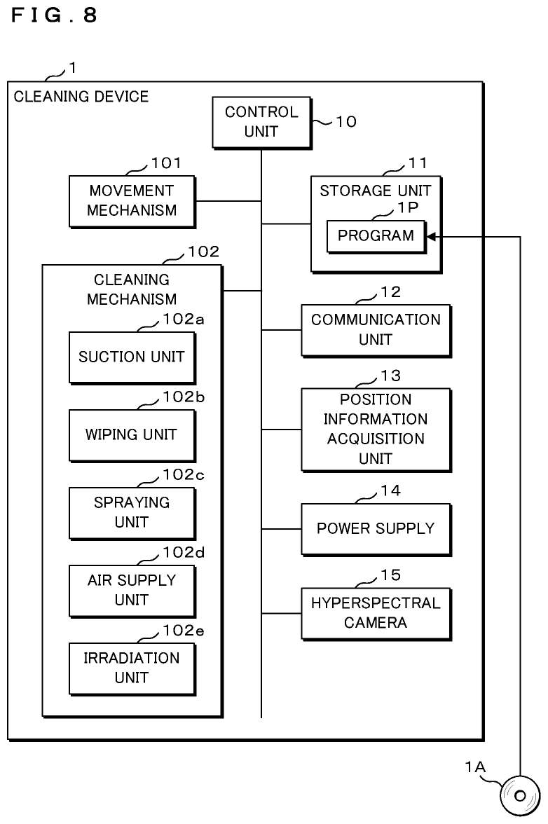

FIG. 8 is a block diagram illustrating the configuration of the cleaning device 1 in the third embodiment. The cleaning device 1 further includes a hyperspectral camera 15. The hyperspectral camera 15 is an imaging device that is capable of dividing a wide wavelength region from a visible region to a near-infrared region into fine wavelength regions, and of measuring light intensity (a spectral value) in each of the wavelength regions. By using the hyperspectral camera 15, it is possible to obtain not only two-dimensional position information but also the spectral value. Molecules configuring a substance perform various movements, and in a case where the moving molecules are irradiated with light, only light having a specific wavelength is absorbed in accordance with a movement state. Accordingly, by acquiring spectral analysis according to imaging data of the hyperspectral camera 15, it is possible to find which molecules a measurement target contains. For example, the moisture has characteristic absorption in the vicinity of 1450 nm, saccharinity has characteristic absorption in the vicinity of 230 nm, protein has characteristic absorption in the vicinity of 280 nm, and a nucleic acid (for example, derived from pathogen such as bacteria or virus, or the like) has characteristic absorption in the vicinity of 260 nm. In addition, hemoglobin in the blood has characteristic absorption in a wide visible region, in the vicinity of 940 nm, and in the vicinity of 660 nm. The cleaning device 1 images the inside of the medical facility by using the hyperspectral camera 15, and acquires the spectral value, thereby specifying the contamination substance existing in the surface to be cleaned. Note that, the hyperspectral camera 15 is not limited to being provided in the cleaning device 1, and for example, may be provided in the medical facility.

In the medical facility that is the cleaning target, the cleaning device 1 images the inside of the medical facility by using the hyperspectral camera 15, and acquires the spectral value, thereby specifying the contamination substance existing in the surface to be cleaned.

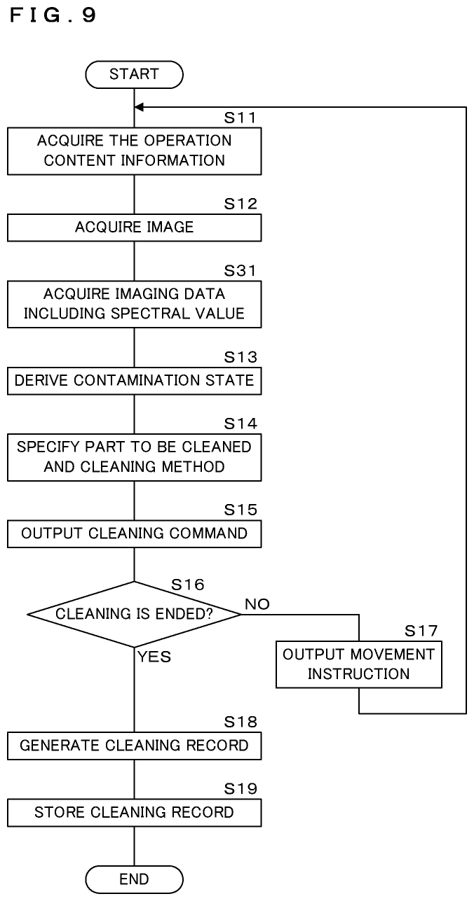

FIG. 9 is a flowchart illustrating an example of a processing procedure that is executed by the cleaning device 1 in the third embodiment. The same step numbers will be applied to the processing common to FIG. 4 of the first embodiment, and the detailed description thereof will be omitted.

The control unit 10 of the cleaning device 1, for example, communicates with the server of the medical facility, thereby acquiring the operation content information (step S11). The control unit 10 acquires the image that is obtained by imaging the inside of the medical facility from the imaging device 2 (step S12).

The control unit 10 images the inside of the medical facility by the hyperspectral camera 15, and acquires the imaging data including the spectral value (step S31). The control unit 10 transitorily stores the acquired operation content information, the acquired image, and the acquired imaging data in association with each other.

The control unit 10 derives the contamination state including the contamination range in the medical facility and the type of contamination substance, on the basis of the acquired operation content information, the acquired image, and the acquired imaging data (step S13). The control unit 10 acquires the attached range of the contamination substance, on the basis of position information of the imaging data obtained by the hyperspectral camera 15, and derives an area corresponding to the acquired attached range as the contamination range by applying the contamination point to the area. In addition, the control unit 10 derives the type of contamination substance corresponding to the position information, on the basis of the acquired spectral value.

The control unit 10 specifies the part to be cleaned in the medical facility and the cleaning method, on the basis of the derived contamination state (step S14). Hereinafter, as with the first embodiment, the control unit 10 executes the processing of step S15 to step S19, and ends a set of processing.

In the processing described above, the control unit 10 may acquire only the imaging data including the spectral value instead of acquiring the operation content information and the image. That is, the control unit 10 may derive the contamination state including the contamination range in the medical facility and the type of contamination substance by using only the imaging data obtained by the hyperspectral camera 15, and may perform the cleaning in accordance with the part to be cleaned and the cleaning method that are specified on the basis of the derived contamination state. The cleaning device 1 may perform the cleaning according to the part to be cleaned and the cleaning method that are specified on the basis of the image of the imaging device 2 or the operation content information, and the cleaning according to the part to be cleaned and the cleaning method that are specified by using the hyperspectral camera 15, independently. The cleaning device 1 may perform the cleaning described in the first embodiment or the second embodiment, and then, may perform the cleaning described in the third embodiment by activating the hyperspectral camera 15. According to this embodiment, the content of the contamination substance in the medical facility is accurately acquired by the hyperspectral camera 15, and thus, the part to be cleaned and the cleaning method can be more effectively specified.

In the examples described in the first embodiment to the third embodiment, other embodiments can be attained by combining all or a part of the configurations described in each of the embodiments.

It is to be noted that, as used herein and in the appended claims, the singular forms “a”, “an”, and “the” include plural referents unless the context clearly dictates otherwise.

The embodiments disclosed herein are an example in all respects, and are not restrictive. The scope of the present invention is indicated by the claims, and includes all changes within the meaning and the scope equivalent to the claims.

What is claimed is:

1-14. (canceled)

15. A non-transitory computer readable recording medium storing a program allowing a computer to execute processing of:
acquiring an image including a still image or a moving image that is obtained by imaging an inside of a medical facility with an imaging device provided in the medical facility, or operation content information relevant to operation content in the medical facility;
specifying a part to be cleaned in the medical facility and a cleaning method, on the basis of the acquired image or the acquired operation content information; and
outputting a cleaning command according to the specified part to be cleaned in the medical facility and the specified cleaning method.

16. The recording medium according to claim 15, the program allowing the computer to further execute processing of
specifying the part to be cleaned including a cleaning route in the medical facility or a part to be intensively cleaned, and the cleaning method including a cleaning means or chemicals to be used, on the basis of the image or the operation content information.

17. The recording medium according to claim 15, the program allowing the computer to further execute processing of:
deriving a contamination range in the medical facility and a type of contamination substance, in accordance with a position or a type of medical service worker performing an operation in the medical facility or a position or a type of goods in the medical facility of the image; and
specifying the part to be cleaned in the medical facility and the cleaning method, on the basis of the derived contamination range in the medical facility and the derived type of contamination substance.

18. The recording medium according to claim 15,
wherein the image includes a medical service worker performing an operation in the medical facility in an imaging target, and
the program allows the computer to further execute processing of:
acquiring an operation state of the medical service worker to be output by inputting the image to a learning model that outputs an operation state of medical service worker when an image that is obtained by imaging the medical service worker performing an operation in a medical facility is input; and
specifying the part to be cleaned in the medical facility and the cleaning method, on the basis of the acquired operation state.

19. The recording medium according to claim 15, the program allowing the computer to further execute processing of:
acquiring a spectral value that is obtained by imaging the inside of the medical facility with a hyperspectral camera; and
specifying the part to be cleaned in the medical facility and the cleaning method, on the basis of the acquired spectral value.

20. The recording medium according to claim 19, the program allowing the computer to further execute processing of:
detecting at least one selected from moisture, saccharinity, protein, and a nucleic acid existing in the medical facility, on the basis of the spectral value; and
specifying the part to be cleaned in the medical facility and the cleaning method, on the basis of a detection result.

21. The recording medium according to claim 15,
wherein the operation content information includes medical chart data relevant to an operation in the medical facility, and
the program allows the computer to further execute processing of:
deriving a contamination range in the medical facility and a type of contamination substance, on the basis of an operation range, an operation content, and goods used, which are specified by the medical chart data; and
specifying the part to be cleaned in the medical facility and the cleaning method, on the basis of the derived contamination range in the medical facility and the derived type of contamination substance.

22. The recording medium according to claim 15, the program allowing the computer to further execute processing of
instructing a movement to a second medical facility different from the medical facility, after cleaning the inside of the medical facility.

23. The recording medium according to claim 22, the program allowing the computer to further execute processing of:
acquiring a second image including a still image or a moving image that is obtained by imaging the inside of the second medical facility with a second imaging device provided in the second medical facility, or second operation content information relevant to operation content in the second medical facility;
specifying a part to be cleaned in the second medical facility and a cleaning method, on the basis of the acquired second image or the acquired second operation content information; and
outputting a second cleaning command according to the specified part to be cleaned in the second medical facility and the specified cleaning method.

24. The recording medium according to claim 23, the program allowing the computer to further execute processing of:
acquiring a second spectral value that is obtained by imaging the inside of the second medical facility with a hyperspectral camera; and
specifying the part to be cleaned in the second medical facility and the cleaning method, on the basis of the acquired second spectral value.

25. The recording medium according to claim 15, the program allowing the computer to further execute processing of
generating a cleaning record including the part to be cleaned in the medical facility and the cleaning method.

26. A cleaning device including a movement mechanism and a cleaning mechanism, the device comprising:
a first acquisition unit acquiring an image including a still image or a moving image that is obtained by imaging an inside of a medical facility with an imaging device provided in the medical facility, or operation content information relevant to operation content in the medical facility;
a first specifying unit specifying a part to be cleaned in the medical facility and a cleaning method, on the basis of the image or the operation content information acquired by the first acquisition unit; and
an output unit outputting a cleaning command according to the part to be cleaned in the medical facility and the cleaning method specified by the first specifying unit,
wherein the part to be cleaned in the medical facility is cleaned by the cleaning method, using the movement mechanism and the cleaning mechanism, on the basis of the cleaning command output by the output unit.

27. The cleaning device according to claim 26, further comprising:
a hyperspectral camera;
a second acquisition unit acquiring a spectral value that is obtained by imaging the inside of the medical facility with the hyperspectral camera; and
a second specifying unit specifying the part to be cleaned in the medical facility and the cleaning method, on the basis of the spectral value acquired by the second acquisition unit.

28. An information processing method, comprising:
acquiring an image including a still image or a moving image that is obtained by imaging an inside of a medical facility with an imaging device provided in the medical facility, or operation content information relevant to operation content in the medical facility;
specifying a part to be cleaned in the medical facility and a cleaning method, on the basis of the acquired image or the acquired operation content information; and
outputting a cleaning command according to the specified part to be cleaned in the medical facility and the specified cleaning method.
